(19) United States
(12) Patent Application Publication
(10) Pub. No.: US 2007/0065527 A1
(43) Pub. Date: Mar. 22, 2007

(54) Activated citrus peel extract

(76) Inventors: Anna Medvedev, Beer Sheva (IL); Efrat Kat, Tel Aviv (IL)

Correspondence Address: NATH & ASSOCIATES, 112 South West Street, Alexandria, VA, 22314, US

(21) Appl. No.: 10/553,288

(22) PCT Filed: Apr. 15, 2004
(86) PCT No.: PCT/IL04/00329

§ 371 Date: Nov. 13, 2006

Related U.S. Application Data
(60) Provisional application No. 60/462,740, filed on Apr. 15, 2003.

(51) Int. Cl. A61K 36/752; A61K 38/16; A61K 31/7048; A61K 31/715; A61K 31/20; A61K 31/22

(52) U.S. Cl. 424/736; 514/2; 514/27; 514/61; 514/558; 514/547

(57) ABSTRACT

The present invention relates to composition comprising an activated citrus peel extract (ACPE) prepared by an activation method which includes exposure of citrus peels to at least one plant or animal pathogen. The ACPE comprises at least one of the following: oligosaccharides, short peptides, flavonoid glycosides, fatty acids, and triglycerides. Compositions comprising the ACPE have been shown useful as dermatological compositions for the treatment of various skin conditions. They have also been shown to be useful in preserving food, beverages and cosmetics.

### FIELD OF THE INVENTION

This invention relates to compositions containing activated citrus peel extract and uses thereof.

### BACKGROUND OF THE INVENTION

Since the beginning of mankind, harvesting of fruits and vegetables has played an important role in everyday life. Modern agriculture has allowed an increase in the amounts of harvested fruits and vegetables and due to lack of appropriate and efficient post-harvesting shipping and storage abilities for large quantities of such fruits and vegetables, has also caused increase in post-harvesting losses, which has become increasingly critical in countries where supply of fruits and vegetables is already low.

Citrus is one such crop. It is actually of the most common crops, and while so, the fruit peels are nearly completely unutilized and therefore discarded. The essential importance of citrus fruit peels has been realized in many fields of research relating to animal and human consumption. Furthermore, the utilization of the citrus fruit peels in the medicinal treatment of both animals and humans has also been realized.

The first thought that comes to mind when citrus fruit peels are mentioned is the oil which may be extracted therefrom by simply squeezing the peels or while the fruit is peeled. The production of this oil for medicinal or religious uses dates back to the eighteen century so that a considerable amount of data has been published about the various extraction methods and the various components identified therein.

Citrus peel extracts have been obtained and used for various applications. These uses are known to depend mostly on the natural activity of the peel extracts against various bacteria and fungi. The efficacy however of the extract on the various applications depends to a large extent on the methods of production. Many processes for the production of the oil have provided extracts which exhibited limited efficacy as the active component existed therein in small quantities.

One method of production of the citrus peel extract is described in Israel Patent No. 120929. This method comprises contacting citrus fruit peels with plant fungal and/or bacterial pathogens, incubating the citrus peels and aqueous extracting from them the active extract.

### SUMMARY OF THE INVENTION

The present invention is directed to compositions containing activated citrus peel extracts, herein designated ACPE, and uses thereof.

The ACPE used in the compositions of the present invention is characterized as being prepared by a method which involves contacting of the citrus peels with at least one pathogen prior to extraction, said method hereon referred to as the “activation method”. The extract obtained from such a method is hereon referred to as the “activated extract” or as ACPE.

The activation method may for example be the method disclosed in Israel Patent No. 120929 or the method of the present invention as disclosed hereinafter. The pathogens utilized for the activation of the citrus peels may be one or more plant or animal pathogens, preferably plant pathogens, selected from fungal or bacterial pathogens. A combination of pathogens may also be used in the activation process. Such combinations may for example be a mixture of plant and animal pathogen, a mixture of two or more different plant pathogens; a mixture of two or more different animal pathogens, a mixture of at least one animal pathogen and at list one animal pathogen, and other similar mixtures. Preferably the pathogens are selected from Penicillium digitatum, Penicillium itallicum, Phytophtora citrophtora and Pseudomonas syringae. Most preferably the pathogen is Penicillium digitatum.

The ACPE is further characterized as comprising one or more of the following: oligosaccharides, short peptides, flavonoid glycosides, fatty acids, and triglycerides. This ACPE has been shown to be effective as antimicrobial and antibacterial agent against a variety of plant or animal pathogens.

In one aspect of the present invention there is provided a composition comprising an activated citrus peel extract (ACPE) prepared by an activation method which includes exposure of citrus peels to at least one pathogen, said ACPE comprising one or more or a combination of all of the following: oligosaccharides, short peptides, flavonoid glycosides, fatty acids and triglycerides.

In one embodiment, the extract comprises at least 30-60% oligosaccharides, 1-10% short peptides, 10-30% flavonoid glycosides, 5-15% fatty acids, and 5-15% triglycerides. In a preferred embodiment, the extract comprises 50-60% oligosaccharides, 3-7% short peptides, 15-25% flavonoid glycosides, 8-12% fatty acids, and 8-12% triglycerides. In a most preferred embodiment, the extract comprises 55% oligosaccharides, 5% short peptides, 20% flavonoid glycosides, 10% fatty acids and 10% triglycerides.

The use of the symbol “%” or the term “percent” in the context of the ACPE extract will be understood to imply a weight proportion of each ingredient contained therein in relation to the weight of the whole extract (100%). For example, “10% fatty acids” refers to an extract containing 10 g fatty acids in every 100 g of extract (w/w proportion).

In one aspect, the composition of the present invention is a dermatological composition and also comprises a dermatologically acceptable carrier, excipient or diluent. The dermatological composition of the present invention may be used for the treatment of skin conditions, which may or may not be associated with a bacterial or a fungal infection. In a preferred embodiment, the composition is adapted for the treatment of a skin condition related to diabetes.

The dermatological composition of the present invention may be used for preventing, alleviating or treating a skin condition. Thus there is provided a method for treatment of a skin condition, said method comprises contacting the skin of a subject in need thereof with an effective amount of the dermatological composition of the present invention, said contacting results in the prevention, alleviation or treatment of the skin condition.

The dermatological compositions may be presented in various forms, preferably as lotions, shampoos, foot, hand and face creams, and soaps.

In another aspect of the present invention, the composition of the present invention is a composition for preserving foods, beverages and cosmetics, said composition comprises said activated citrus peel extract (ACPE).

The foods and beverages which may be preserved by the composition of the present invention are for example meats, dairy products, water, soups, pastes, vegetable and fruit juices, chocolates, snacks, confectionery, flour based foods, tea, coffee, alcoholic and carbonated beverages, vitamin complexes and health foods.

In yet another aspect of the present invention there is provided a biocide composition which comprises ACPE for cleaning and disinfecting surfaces such as those found in households, hospitals, poultry and animal husbandry.

In a further aspect of the present invention there is provided a process for the preparation of an activated citrus peel extract (ACPE). The process comprises:
(i) contacting citrus peels with spores of at least one fungal or bacterial pathogens, said pathogens being a 16 hour to 24 hour old bacteria or a 8 day to 14 old fungus,
(ii) incubating said citrus peels;
(iii) extracting the peels with water, and removing the peels from the aqueous liquid to obtain an aqueous extract.

In one embodiment, the process may include the adjustment of the pH of said un-concentrated aqueous extract obtained in step (iii) to a first pH of 8-10, filtrating it through a membrane having cutoff of between 800-2000 Da, re-adjusting its pH to second pH of 3-5, and concentrating the filtrate to obtain said activated citrus peel extract.

In another embodiment, the process may include the step of filtration of the extract of step (iii) through a membrane having cutoff of between 800-2000 Da without prior pH adjustment.

In a most preferred embodiment the process comprises the following:
(i) contacting citrus peels with spores of at least one fungal or bacterial pathogens, said pathogens being a 16 hour to 24 hour old bacteria or a 8 day to 14 old fungus,
(ii) incubating said citrus peels;
(iii) extracting the peels with water, and removing the peels from the aqueous liquid thereby obtaining an aqueous extract;
(iv) adjusting the pH of said aqueous extract obtained in step (iii) to a first pH, filtering the solution through a membrane having a cutoff between 800-2000 Da, readjusting the pH to a second pH and concentrating the filtrate to obtain said activated citrus peel extract.

In one case, the pathogens are selected from Penicillium digitatum, Penicillium itallicum, Phytophtora citrophtora and Pseudomonas syringae. The pathogen is preferably Penicillium digitatum.

In yet another aspect the present invention provides an activated citrus peel extract (ACPE) obtainable by the method of the present invention.

In yet another aspect the present invention provides an activated citrus peel extract (ACPE) obtained by the method of the present invention.

Also comprised within the scope of the present invention are the uses of the ACPE in the preparation of said compositions and methods for the treatment of skin conditions.

### DETAILED DESCRIPTION OF THE INVENTION

According to one aspect of the present invention, there is provided a composition comprising an ACPE produced by a method which involves activation of the citrus peels prior to the extraction process, and which results in an extract or active mixture having the following ingredients: oligosaccharides, short peptides, flavonoid glycosides, fatty acids and triglycerides.

Extract composition analysis utilizing separation methods such as various chromatographic separations and others known in the art, of various lots of extracts obtained under similar activation conditions showed variations in the relative quantity of each ingredient. All compositions showed identical or closely similar activity, both in terms of efficacy and selectivity towards certain microorganisms. Examples of such extract compositions are as follows:

1. 55% oligosaccharides, 5% short peptides, 20% flavonoid glycosides, 10% fatty acids and 10% triglycerides;

2. 46% oligosaccharides, 3% short peptides, 18% flavonoid glycosides, 16% fatty acids, and 17% triglycerides;

3. 35% oligosaccharides, 10% short peptides, 30% flavonoid glycosides, 10% fatty acids, and 15% triglycerides;

4. 60% oligosaccharides, 6% short peptides, 25% flavonoid glycosides, 5% fatty acids and 4% triglycerides;

5. 41% oligosaccharides, 10% short peptides, 19% flavonoid glycosides, 15% fatty acids, 12% triglycerides and 3% unidentified components;

6. 51% oligosaccharides, 9% short peptides, 25% flavonoid glycosides, 5% fatty acids and triglycerides (combined) and 10% unidentified components.

The term “comprise” or variations thereof will be understood to imply the inclusion of a stated integer or a group of integers but not the exclusion of any other integer or group of integers. The term “composition” as used within the scope of the present invention, refers to a composition which includes the ACPE and may also include other integers. The ACPE may include other integers which may originate from the type of citrus used, the age of the peels, freshness of the peels, possible prior exposure of the fruit and peels (before harvesting of the fruit) to natural pathogens, the type of pathogen used for the activation process, its age, period of exposure and other variables. Nevertheless, these other ingredients do not affect in any way the activity of the ACPE as herein described and exemplified. Specifically, an expression such as “ACPE comprises one or more of the following: oligosaccharides, short peptides, flavonoid glycosides, fatty acids, and triglycerides” will be understood to imply an extract that despite the exclusion of one or more of the listed integers or despite the inclusion of other integers, maintains its antibacterial or antimicrobial activity and exhibits no lowered or diminished such activity.

Notwithstanding the above, typically the composition of the present invention will comprise a combination of all of the hereinbefore listed integers.

The term “Oligosaccharides” refers to a sugar containing 8 to 15 monosaccharide units joint by glycosidic bonds.

The term “peptide” refers to compounds made up of two or more amino acids joint by covalent bonds. The term “short peptides” refers to peptides having at least 2 such amino acids and having molecular weights of less than 800 g/mole.

The term “flavonoid” refers generally to compounds having a C6-C3-C6 ring structure that is two aromatic rings linked together with a C3 segment which in most cases constructs a phenyl-benzpyran skeleton. Within the context of the present invention, the term includes also subclasses such as flavones, flavonols, flavanones, flavanols, anthocyanidines, isoflavonoids, and derivatives thereof. The flavonoids may or may not have hydroxyl groups and glycosidic linkages. Those having such linkages are referred to as “flavonoid glycosides”. Those lacking sugars are termed “flavonoid aglycones” and are too encompassed within the present definition. Also encompassed with in the term are open ring compounds that structurally maintain the benzpyran skeleton.

Non-limiting example of flavonoids are: apigenin, luteolin and luteolin-7-glycosides, artemetin, casticin, 5-hydroxy-3,6,7,4′-tetramethoxyflavone, rutin, tanetin, vitexin, hesperidin, naingin, hesperetin and naringenin.

The term “fatty acid” refers to saturated or unsaturated organic acids having more than 4 carbon atoms and one carboxylic acid group. Non-limiting examples of fatty acids are: linoleic acid, myristic acid, oleic acid, palmitic acid, and stearic acid. The term “triglyceride” refers to fatty acid triesters of glycerol. The triglycerides may be of the same fatty acid or a mixture thereof and may or may not be fully substituted.

The expression “at least one pathogen” or “at least one fungal or bacterial pathogen” refers to pathogens that may be utilized for the activation of the citrus peels. These may be one or more plant or animal pathogens, preferably plant pathogens, selected from fungal or bacterial pathogens. A combination of pathogens may also be used in the activation process. Such combinations may be a mixture of plant and animal pathogen, a mixture of two or more different plant pathogens, a mixture of two or more different animal pathogens, a mixture of at least one animal pathogen and at list one animal pathogen, and other similar mixtures. Preferably the pathogens are selected from Penicillium digitatum, Penicillium itallicum, Phytophtora citrophtora and Pseudomonas syringae. The pathogen is most preferably Penicillium digitatum.

According to one embodiment, the composition is a dermatological composition for the treatment, alleviation and/or prevention of various skin conditions.

The terms “prevention”, “alleviation” and “treatment” as used herein refer to the administering of an amount of the composition of the present invention which is effective to ameliorate undesired symptoms associated with a condition, to prevent the manifestation of such symptoms before they occur, to slow down the progression of the condition, slow down the deterioration of symptoms, to enhance the onset of remission period, slow down the irreversible damage caused by the condition, to delay the onset of said progressive stage, to lessen the severity or cure the condition, or to prevent the condition form occurring or a combination of two or more of the above.

In one embodiment, the skin condition may be associated with a bacterial or a fungal infection. In another embodiment, the skin condition may be a secondary condition, resulting from diseases and conditions not associated with a bacterial or a fungal infection of the skin. Secondary conditions may for example be wounds caused by such infections and other secondary conditions as mentioned hereinafter.

The term “bacterial infection” refers to a skin infection caused by one or more bacteria, such as: Propionibacterium acnes, Entrococcus, hemolytic Streptococci, Staphylococci and M.R.S.A. (Methicillin resistant Staphylococcus aureus), or a combination thereof. Examples of bacterial skin conditions in humans or animals are, without being limited to, acne, cellulites, folliculitis, boils (or carbuncles), Staphylococcal scalded skin syndrome, Erysipelas, Erythrasma, Impetigo and Paronychia.

The term “fungal infection” refers to a skin infection caused by one or more fungi such as Canis, Trichophyton, Mentagraphtes, Rubrum, Violaceum, Epidermophyton, Icrosporum and Candida, or a combination thereof. Examples of skin conditions associated with fungal infections are, without being limited to, ringworm, Candidiasis, Tinea Pedis and Tinea versicolor.

In one specific case, the dermatological composition may be used for the treatment of a skin condition associated with a combination of a bacterial and a fungal skin infection.

A composition comprising ACPE was tested on a number of common fingi isolated from human individuals and which belonged to the Trichophyton group: Mentagraphytes, Rubrum, and Violaceum. The inhibition of the fungi by ACPE was tested as described in the examples below. The composition was shown to inhibit all three fungi and also inhibited a Microsporum Canis (a fungus from the Canis group).

In another embodiment, the dermatological composition is used for the treatment of skin conditions that are not directly caused by a bacterial or a fungal infection. Such conditions are for example, without being limiting to, diabetes related skin conditions, skin injuries, dermatitis, bedsores, dry skin, celluses, corns, Keratosis Pilaris, psoriasis, pityriasis rosea and rosacea.

The composition is preferably used for treating skin condition related to diabetes. An ACPE composition comprising 0.3 g/ml of ACPE in water was tested on 10 human individuals who observed open skin wounds resulting from active diabetes. ACPE was spread on the affected skin twice a day for a period of one week. Within 4 to 5 days after first administration of the ACPE, full healing of the broken skin was observed.

The term “dermatological composition” refers to a composition containing ACPE and which is capable of assisting in the treatment of a skin condition and for the regeneration of skin cells, when applied to the skin. The term also encompasses a cosmetic preparation designed to beautify the body by direct application of the composition to the skin. In this respect, the term “skin” encompasses whole skin or any portion of the human or animal skin, including hair, nails, etc.

Dermatological compositions of the present invention may be prepared with the ACPE for various types of applications such as those known to a person skilled in the art. However, preferably the compositions are prepared for topical application. For this purpose, standard formulations such as creams, drops, ointments, gels, lotions or compositions for masks into which the ACPE may be worked as a solution, a lyophilisate, a suspension or an emulsion may be used. The amount of ACPE used is sufficient to achieve the desired effect.

In treating widespread skin condition the treatment may require the soaking of cloths or bandages in a solution containing ACPE and the appropriate carrier, excipient or diluent and the application of the cloth or bandage to the diseases area of the skin. Such cloths or bandages may be made from any material capable of absorbing and maintaining a moist environment for the skin site.

The dermatologic compositions may be based on conventional carrier systems used for topical applications such as polyethylene glycols, carboxymethyl cellulose, carboxyvinyl polymerisates, paraffin oil (liquid paraffin), cetylstearyl alcohol, fatty acid triglycerides, oleic acid ester, polyacrylates, glycerol, alcohols and the like or mixtures thereof. They may further contain auxiliary agents such as preservatives, perfume oils, buffers, wetting agents and the like.

The dermatologic composition may be in the form of soaps, lotions or creams such as hand-, foot- and face-creams. The composition may also be in the form of a shampoo for the treatment of the scalp and hair. The shampoo composition may contain, in combination with the ACPE, at least one anionic, cationic, nonionic or amphoteric detergent, suitable for use in hair treatment.

For Example, the anionic detergents include, among others, alkyl sulfates, alkylether sulfates, alkylpolyether sulfates, alkyl sulfonates, monoglyceride sulfates, alkanolamide sulfates, alkanolamide sulfones, soaps of fatty acids, the condensation products of a fatty acid with isethionic acid, the condensation product of fatty acids with methyl taurine, the condensation products of fatty acids with sarcosine and the condensation products of fatty acids with a protein hydrolyzate.

The cationic detergents include, among others, long chain quaternary ammoniums, such as dilauryldimethyl ammonium chloride, diisobutyl phenoxyethoxy ethyl dimethylbenzyl ammonium chloride, cetyl trimethyl ammonium bromide, N-cetyl pyridinium bromide and benzethonium chloride, lauryl benzyl trimethyl ammonium bromide or chloride, myristyl benzyl trimethyl ammonium bromide or chloride and cetyl benzyl trimethyl ammonium bromide. Other cationic detergents may be esters of fatty acids and amino alcohols and polyetheramines.

Nonionic detergents may be selected from esters of polyols and sugars, the condensation products of ethylene oxide on fatty acids, on fatty alcohols, on long chain alkylphenols, on long chain mercaptanes, on long chain amides, and polyethers of polyhydroxylated fatty alcohols. Suitable amphoteric detergents include asparagines derivatives and alkylamino propionates.

These shampoo compositions may also be in the form of a dry powder and may further contain conventional cosmetic components as perfumes or dyes typically used in the shampoo industry.

The invention also concerns the use of the ACPE for the preparation of a dermatologic composition comprising a dermatologically acceptable carrier, excipient or diluent and an extract produced by contacting citrus fruit peels with fungal or bacterial pathogens, having the ingredients composition disclosed hereinbefore.

The dermatological composition of the present invention may be applied in a sequential manner until the skin condition is resolved or under control. The cosmetic compositions may be formulated in such a way as to provide any desired release profile, including fast, sustained or delayed release of the ACPE after the initial administration to the individual, by employing any of the procedures known in the art.

The composition may also be used in combination with other known compositions and/or other effective medicaments to increase or enhance the effect on the skin.

The compositions of the present invention are not harmful to the body of either humans or animals and thus varying concentrations of compositions may be used to achieve the desired effect.

According to this aspect of the invention, there is also provided a method of producing the composition for dermatologic use. This method comprises adding the ACPE having the constituents listed hereinbefore to a dermatologically acceptable carrier such as those described before. The addition may be performed during the manufacture of the composition or may be performed immediately prior to use when the ACPE and the carrier are sold separately.

In one preferred embodiment, the composition is prepared by mixing or diluting the ACPE with a carrier, which may be a solid, a semi-solid, or a liquid acting as a vehicle, excipient or medium for the ACPE.

For example, an ACPE active composition may be prepared by adding 10 g of ACPE to 90 g of a pre-mixed cream having one or more of the following non-active ingredients: mineral spring water, mallow extract, chamomile extract, cetyl alcohol, petrolatum, plantain extract, propylene glycol, isopropyl myristate, urea, clycerin, aloe vera-gel, olive oil, isopropyl plamitate, evening primrose oil, sweet almond oil, jojoba oil, wheat germ oil, grape seed oil, avocado oil, fragrance, D & C red no. 4, FD & C blue no. 1, or any other FDA approved coloring agent.

In another preferred embodiment, the composition is used as a cleansing agent and may further contain neutralizing additives such as sodium bicarbonate and ammonia.

According to another aspect of the invention, the ACPE composition is a composition for preserving foods, beverages, cosmetics, or any other composition, substance or utility which may be susceptible to spoilage or decomposition as a result from exposure to various microorganisms, including bacteria, fingi, and the like. The composition comprises the ACPE of the present invention, having the constitution disclosed hereinbefore, and an acceptable carrier, excipient or diluent which is chosen in line with the expected utility of the composition.

In one embodiment, the preservative is added to foods and beverages for extending shelf-life and protect from natural or imposed deterioration caused by such elements as microorganisms, natural food enzymes, insects and the like, varying temperatures, air and light mediated oxidations, variations in humidity and time. Such foods and beverages may for example be, without limiting thereto, fruits including dried fruits, vegetables including leaf vegetables and root crops, vegetable and fruit juices, fish, meats and dairy products, water, soups, pastes, chocolates, snacks, confectionery, flour based foods, tea and coffee, alcoholic beverages, carbonated beverages, vitamin complexes and health foods.

In case of fruits, vegetables and other crops, the composition containing ACPE may be used for extending post-harvest shelf-life utilizing technologies that are part of the development process in agriculture.

Thus, there is provided a solution comprising the ACPE for extending shelf-life of whole fruits and vegetables, said solution is composed of an effective amount of ACPE and water or other suitable liquid or solid media.

The solution for extending shelf life of fruits and vegetables may be applied to fruits and vegetables by any means including wetting, washing, spray, immersion, and the like.

The solution of the present invention may also be incorporated into wrapping or containing materials such as those used to contain or hold fruits and vegetables. The ACPE composition contained within said wrapping or containing materials may be designed such as to allow slow or controlled release of the active extract from the wrapper or the container to the fruit or vegetable contained therein during storage or shipment. The wrapping or containing materials may for example be pouches, plastic or paper bags, nylon sheets, polyester sheets, paper wrapping, plastic or other sealed containers, paper or plastic materials for hand or machine wrappings of fruits and vegetables, and the like.

Examples of whole fruits and vegetables that may be treated for prolonging their shelf life are: citrus fruits, tomatoes, grapes, peaches, bananas, mangos, apricots, pears, potatoes, cucumbers, carrots, eggplants, peppers, radishes, tobacco leaves, spinach leaves, lettuce, cherries, apples, papayas, plums and the like.

In another embodiment, the preservative may be used to prolong the shelf-life of cosmetics, such as, but not limiting to, creams, ointments, gels, lotions, compositions for face masks and the like.

For prolonging produce shelf life, the ACPE preservative may be added during the preparation or treatment of the produce in sufficient amounts, thereby inhibiting or minimizing growth of microorganisms.

The preservative composition as with the other composition disclosed herein are able of being effective against a wide range of microorganisms such as Salmonella sp., Staphylococci sp., Streptococci sp., Micrococci sp., E. Coli, Coliform species, Pseudomonas sp., Entrococci sp., Pasteurella sp., Alternaria spp., Fusarium spp., Penicillium spp., Cladosporium spp., Botrytis cinerea, Aspergillus niger, and others hereinbefore and in the examples which follow.

In a further aspect of the present invention, the ACPE composition is a biocide composition for cleaning and disinfecting which comprises a surfactant and the ACPE of the present invention.

The term “biocide composition” refers to a composition containing ACPE which is capable of destroying a whole population of living microorganisms or any portion thereof. The term encompasses also disinfecting and sterilizing capabilities.

The surfactant is preferably selected from nonionic and cationic surfactants. The nonionic surfactant may, for example, be one or more selected from polyglycol ethers, polyalkylene glycol dialkyl ethers, and the addition products of alcohols with ethylene oxides and propylene oxides.

The cationic surfactant may be selected from various quaternary ammonium salts such as, but not limiting to octyl dimethyl benzyl ammonium chloride, octyl decyl dimethyl ammonium chloride, dioctyl dimethyl ammonium chloride, didecyl dimethyl ammonium chloride and dimethyl ethyl benzyl ammonium chloride, or mixtures thereof such as, but not limiting to, alkyl dimethyl benzyl ammonium chlorides and dialkyl dimethyl ammonium chlorides.

In one embodiment, the biocide composition may further comprise dyestuffs, perfumes, builders, chelating agents and corrosion inhibitors. The composition may be used to clean and disinfect surfaces such as ceramic tiles, PVC, porcelain, stainless steel, marble, silver and chrome to remove grease, wax, oil, dry paint and mildew and the like. As it has been demonstrated herein the ACPE containing compositions are non-toxic to the human skin or body. The detergent composition may therefore also be used as a laundry additive.

In another embodiment, the detergent is used in poultry and animal husbandry. As this detergent composition is able of being effective against a wide range of microorganisms such as Salmonella sp., Staphylococci sp., Streptococci sp., Micrococci sp., E. Coli, Coliform species, Pseudomonas sp., Entrococci sp., Pasteurella sp., Alternaria spp., Fusarium spp., Penicillium spp., Cladosporium spp., Botiytis cinerea, Aspergillus niger, it may be used as a one-detergent substitute for several conventional detergent compositions.

The biocide composition may be in a liquid or solid form depending on the specific utility. The detergent may also take the form of an aerosol spray, in which case, the composition is mixed with an appropriate propellant such as mist activators and sealed in an aerosol container under pressure.

In one specific embodiment, the biocide composition is absorbed in a towel or a cloth, thus providing a disinfectant towel that may be used as means of applying the composition to the various surfaces or may be used to disinfect the hands and skin of an individual.

The biocide composition may be further used for the treatment of water reservoirs such as, but not limiting to, water systems, cooling systems, swimming pools, natural and artificial water reservoirs, fisheries, water tanks, aquariums, and any other volume of water.

In one embodiment, the composition is added in a dry form to the water reservoir in an amount sufficient to control the growth of bacteria and fungi. In another embodiment, the dry composition is added to a water reservoir after being dissolved in an appropriate vehicle.

In addition to the antibacterial and antifingal uses described hereinbefore, the composition comprising ACPE, namely, the dermatological and cosmetic composition, and the composition for preserving foods, vegetables and cosmetics and the compositions for prolonging shelf-life thereof may also be used as an antioxidant or in the case of the dermatological composition also as a cleansing agent.

In accordance with yet another aspect of the present invention, and as already disclosed hereinabove, it has been found that a more efficient ACPE extract may be obtained when applying the following procedure. Whole fruit was washed with water and other suitable detergents and then dried. The dried fruits were next peeled, cut to desired size, placed in incubation containers and sprayed with a pathogen, preferably being a plant pathogen, most preferably being a 16-hour to 24-hour old bacteria or 8 to 14 day-old fungus suspension. In case of a fungus suspension, the spore concentration is preferably 4×10⁷ spores/ml. The containers were then sealed and left at room temperature for 4 days. The peels were then transferred into a heating vessel and heated in water at 70° C. for 2 hours. Then, the aqueous solution was run through a colander and the peels were pressed to collect the extract.

At this stage in the process, excess water may be removed by evaporation, yielding an opaque extract having the desired activity as herein described. This extract may however be improved by treating the water solution, prior to evaporation of the water.

Thus, the pH of the aqueous solution was made basic and was next loaded on a filtration unit, preferably an ultra-filtration unit, fitted with a membrane having a cutoff of 800 to 2000 Da. The preferred membrane was one having a cutoff of 1000 Da. A membrane having a cutoff of 1000 Da refers to a membrane that allows passage of molecules having molecular weight smaller or equal to 1000 Da. Molecules with higher molecular weights would not pass the membrane and would therefore remain collected thereon.

It should be pointed out that such a filtration may be performed with any filtration unit known in the art and which a person skilled in the art would find suitable for the purpose of this procedure.

After 7 hours the filtration ended, the pH of the collected filtrate was reduced to the desired pH and the solution was concentrated to obtain the desired ACPE.

The high molecular weight fraction, which was left behind, showed no activity. The ACPE, on the other hand, exhibited all the required characteristics.

The plant pathogens, which may be used in the process, may be anyone selected from a group of non-toxic pathogens. Preferably the pathogens are Penicillium digitatum, Penicillium itallicum, Phytophtora citrophtora and Pseudomonas syringae. Most preferably the pathogen is Penicillium digitatum.

The process may comprise a further step of sterilization and pasteurization of the extract obtained thereby. The sterilization or pasteurization may be on the aqueous solution containing the ACPE or on the ACPE itself and may follow procedures well known in the art.

As many varying and different embodiments and many different modifications may be made to the different embodiments within the scope of the present inventive concept, it is to be understood that the details provided hereinbefore and hereinafter are to be taken in the broader sense as an illustration of the inventive concept and not in a limiting sense.

### EXAMPLES

The following examples are intended to further illustrate the present invention without limiting the scope thereof as claimed.

### Example 1

### A Method for the Preparation of an Activated Citrus Peel Extract (ACPE)

The ACPE utilized in the compositions of the present invention may be prepared, for example by the method of Israel Patent No. 120929 or by the process of the present invention. The process of the present invention involves the following steps:
1. Whole fruit is washed with water, 70% sodium hypochlorite, and ethanol, rinsed again with water and then dried.
2. The dried fruits are next peeled, cut to desired size and placed in incubation containers.
3. Spore concentration of 4×10⁷ spores/ml (filtered spores) of 10 day-old Penicillium digitatum are sprayed onto the peels in a homogenous fashion and the containers are then sealed and left at room temperature (25° C.) for 4 days.
4. Next, the peels were transferred into a heating vessel and were heated in water (5:1) at 70° C. for 2 hours. Then, the aqueous solution was run through a colander and the peels were pressed to collect the extract.
5. The pH of the aqueous solution was now raised to 8 and was next loaded on an ultrafiltartion unit fitted with a membrane having a cutoff of 1000 Da. After the filtration ended, the pH of the collected filtrate was reduced to 3.5 and the solution was concentrated to obtain the desired ACPE.

### Example 2

### Activity of the ACPE in Comparison to Other Citrus Peel Extracts

The activity of the ACPE in inhibiting microbial and fungal growths was examined in comparison with the activity of other citrus peel extracts that were obtained by the following methods:

Method 1: Extraction with Ethanol:

Ethanol (95%, 2.5 L) was added to 500 grams of fresh grapefruit peels. The peels were extracted at 60° C. for 5 hours. The extract thus obtained was filtered through a common colander and the filtrate was concentrated under vacuum to obtain 150 grams of a syrupy extract.

Method 2: Extraction with Propylene Glycol:

The method described in Method 1 above utilizing a 10% solution of Propylene glycol in water (2.5 L).

Method 3: Extraction with Glycerol:

The method described in Method 1 above utilizing 10% glycerol in water (2.5 L).

Method 4: Extraction with Water:

500 grams of fresh grapefruit peels were extracted in 2.5 L water at 70° C. for 2 hours. The extract thus obtained was filtered through a common colander and the filtrate was concentrated under vacuum to obtain 150 grams of a syrupy extract.

Method 5: Extraction Using Cold Press:

The citrus extract was obtained by cold press according to known methods in the industry, producing an extract containing 37.4% TSS (Total soluble solids).

The activity of each of the citrus peel extracts obtained by any one of the above methods was compared with the ACPE extract according to the following “disc” method:

Disc Method: Differing quantities of the ACPE were dripped onto 13-mm paper discs and were then dried. The discs now absorbed with the ACPE were placed at the center of a Petri dish in which a specific microorganism was grown. After 24 hours, the radius of the growth-inhibited area was measured (in mm) and recorded. After an additional 24 hours the measurement was repeated.

Distilled water was used as a negative control. As Table 1 shows, the activity of the ACPE against bacterial and fungal growth was enhanced in comparison with the activity of other citrus extracts obtained with un-activated methods.

TABLE 1
Activity of ACPE as compared to other citrus peel extracts.

| | | |
|---|---|---|
| Method/Inhibition by ACPE, in mm | E-Coli | Cladosporium |

| | | |
|---|---|---|
| 1 | 4.8 | 1.5 |
| 2 | 4.5 | 1.7 |
| 3 | 2.5 | 1.8 |
| 4 | 3.8 | 2.2 |
| 5 | 2.5 | 2.3 |
| ACPE | 17.4 | 10.2 |

The activity of the ACPE obtained by the process of the present invention was also compared to the activity of the extract obtained by the process of Israel Patent No. 120929. A Serial dilution test showed that the ACPE prepared by the method of Example 1 was 4 times as active against Cladosporium as was the extract of Israel patent no. 120929 and twice as active against E. coli as compared to the extract of patent no. 120929.

### Example 3

### Preparation of ACPE Containing Dermatological Formulation I

A formulation was prepared by admixing:

| | |
|---|---|
| ACPE | 50-99% |
| Glycerol | 1-50% |
| Perfume | 1% or less |

### Example 4

### Preparation of ACPE Containing Dermatological Formulation II

A formulation was prepared by admixing the following ingredients:

| | |
|---|---|
| ACPE | 10% |
| Cream | 90% |

The cream used contained: mineral spring water, mallow extract, chamomile extract, cetyl alcohol, petrolatum, plantain extract, propylene glycol, isopropyl myristate, urea, clycerin, aloe vera gel, olive oil, isopropyl plamitate, evening primrose oil, sweet almond oil, jojoba oil, wheat germ oil, grape seed oil, avocado oil, fragrance, D & C red no. 4, FD & C blue no. 1, methyl paraben and/or propyl paraben. This cosmetic lotion was tested as described in Example 7 below.

### Example 5

### Preparation of ACPE Containing Dermatological Formulation III

A formulation was prepared by admixing the following ingredients:

| | |
|---|---|
| Deionized water | 10.00% |
| ACPE extract (from Grapefruit only) | 50.00% |
| Glycerine | 2.00% |
| Sodium lauryl sulphate | 2.00% |
| Sodium laureth sulphate | 15.00% |
| Chamomile extract | 2.00% |
| Coco amido propyl betain | 4.00% |
| Coco amide DEA | 4.00% |
| Aloe vera gel | 3.60% |
| Vitamine E | 0.39% |
| Sodium chloride | 3.00% |
| Methyl paraben | 0.05% |
| Propyl paraben | 0.05% |
| Titanium oxide | 4.00% |

### Example 6

### Preparation of ACPE Containing Dermatological Formulation IV

A formulation was prepared by admixing the following ingredients:

| | |
|---|---|
| Deionized water | 2.80% |
| ACPE extract (from Grapefruit only) | 50.00% |
| Glycerin | 2.00% |
| Sodium lauryl sulphate | 2.00% |
| Sodium laureth sulphate | 15.00% |
| Chamomile extract | 2.00% |
| Eucalyptus oil | 0.40% |
| Rosmarine Oil | 0.50% |
| Tea tree oil | 2.00% |
| Lavender oil | 0.30% |
| Sage extract | 1.00% |
| Rosmarine extract | 1.00% |
| Coco amido propyl betain | 4.00% |
| Coco amide DEA | 4.00% |
| Aloe Vera gel | 3.60% |
| Vitamin E | 0.39% |
| Sodium chloride | 3.00% |
| Methyl paraben | 0.05% |
| Propyl paraben | 0.05% |
| Titanium oxide | 4.00% |
| Sage oil | 1.00% |
| Pine tree oil | 0.50% |
| Lemon oil | 0.50% |

### Example 7

### Use of ACPE as Antimicrobial Component in Cosmetic Products

The formulation of Example 4 was tested on dermathophytal pathogens and the results were compared with the same cream that contained no ACPE. The results (not shown) indicated that the ACPE containing formulation was highly efficacious in a number of cases involving skin fungi, principally against species that belong to the genus Candida and Trichophyton, in comparison with the non-active formulation.

### Example 8

### ACPE Activity Against Microorganisms in Aqueous Solution

In this experiment, 100 g aqueous samples containing each 50% ACPE (w/v) were separately inoculated by one of the test organisms shown in Table 2. The inoculated containers were incubated at 25° C. together with the un-inoculated samples that contained water only.

The number of surviving microorganisms was monitored periodically during a 4-week incubation period.

TABLE 2
Preservative effectiveness test results.

| | | Before Inoculation | Initial Contamination | No. of Surviving Microorganisms CFU/gr | | | | |
|---|---|---|---|---|---|---|---|---|
| Test Organisms | | CFU/gr | CFU/gr | 1 day | 1 week | 2 weeks | 3 weeks | 4 weeks |
| E. coli | 8739 | <10 | 1.8 × 10⁵ | <10 | <10 | <10 | <10 | <10 |
| Staphyl. aureus | 6538 | <10 | 1.3 × 10⁵ | <10 | <10 | <10 | <10 | <10 |
| Ps. aeruginosa | 9027 | <10 | 1.6 × 10⁵ | <10 | <10 | <10 | <10 | <10 |
| Cd. albicans | 10231 | <10 | 1.8 × 10⁵ | 5.6 × 10⁴ | <10 | <10 | <10 | <10 |
| Asp. niger | 16404 | <10 | 1.3 × 10⁵ | 7.8 × 10⁴ | 3.5 × 10³ | 350 | 10 | <10 |

| | | | | | | | |
|---|---|---|---|---|---|---|---|
| Uninoculated Control | <10 | — | <10 | <10 | <10 | <10 | <10 |

As Table 2 shows, the efficiency of the ACPE in controlling the growth of the tested microorganisms was high. In the case of E. Coli, Staphylacoccus aureus and Pseudomonas aeruginosa the ACPE completely inhibited growth within the first day of inoculation. With Cd. Albicans and Asp. niger, inhibition progressed over a period of 1-2 weeks.

### Example 9

### ACPE as a Preservative of Dermatological Formulations

Three different types of cosmetic cream having the hereinbefore exemplified ingredients (which previously did not contain any ACPE or other preservatives such as methyl paraben or propyl paraben) were mixed with 10-20% ACPE. Creams that did not contain any ACPE were used as controls. The controls were contaminated with fungus after several weeks. Creams which contained 10-20% ACPE were stable and showed no fingi related contamination for periods of several months (more than 3 months).

### Example 10

### Use of ACPE for the Treatment of Fungal-Associated Skin Conditions

ACPE was tested on a number of common fungi isolated from human individuals. The fungi belong to the Trichophyton group: Mentagraphytes, Rubrum, and Volaceum. The inhibition of the fingi by ACPE was tested as described in Example 2. ACPE was shown to inhibit all three fungi as shown in Table 3. A fungus from the Canis group, Microsporum Canis, was shown to be inhibited by the ACPE as well (results not shown). As a control, paper discs with distilled water were used and showed no inhibition.

TABLE 3
Inhibition of human fungi.

| Fungi tested | Inhibition by ACPE, in mm |
|---|---|
| Mentagraphytes | 12 |
| Rubrum | 16 |
| Violaceum | 22 |
| Control | 0 |

### Example 11

### Preparation of a Skin Patch Containing ACPE

A skin patch for the treatment of a skin condition is prepared by soaking a sterile patch or bandage in a solution containing sterile ACPE and sterile natural oil such as a paraffin oil in a ratio of about 1:15 and an emulsifier such as Twin 20. The soaked patch or bandage is then directly used on the skin or is packed under sterile conditions to be opened on need and placed on the area of the skin requiring treatment.

### Example 12

### ACPE Activity as an Antimicrobial Agent

The evaluation of the effectiveness of the ACPE against Staphylococcus aureus, Streptococcus pyogenes and Trichophyton rubrum (wild strain) was conducted at ACPE concentrations of 1%, 10%, 50% and 100% and at pH 3.5 and pH 5.

The sample dilutions were inoculated each one separately with a microbial load of 10⁵ cfu/ml of the challenge organisms, according to the guidelines set out in “Testing organisms and Preparation of Inoculum” USP 26.

The inoculated specimens were allowed to stand at room temperature for 6 and 24 hour periods. At the end of each period, 100 μl aliquots of each inoculated tube were transferred to an appropriate medium in separate Petri dishes. The media used were: Tryptic Soy Agar (TSA), Sabourand Dextrose Agar (SDA) plus Choramphenicol and Sterile Saline Solution. The TSA and SDA were also used for monitoring.

The number of cfu present in each test sample for the intervals was determined by the plate count procedure known to a person skilled in the art. By using the calculated concentrations of cfu per ml present at the start of the test, the change in log₁₀ values of the concentration of cfu per ml for each microorganism was calculated. This change is expressed below in terms of log reductions; for example, a −5 log₁₀ reduction would mean a reduction of five orders of magnitude, for example from the 10⁵ microorganisms per ml starting load to less than 10 microorganism per ml. Such a log reduction is considered as a total eradication of the microorganisms' population and a total inhibition of any further growth.

For Streptococcus pyogenes, test solutions at pH 3.5 showed a total eradication of the microorganism's population. A −5 log₁₀ reduction was exhibited for the initial calculated count at all concentrations at both 6 and 24 hours and was also achieved with the pH 5.0 up to the 10% solution concentration. The 1% solution showed a reduction of −1.64 log₁₀ after 6 hours and of −5 log₁₀ after 24 hours.

For Staphylococcus aureus, test solutions containing 10%, 50% and 100% ACPE at pH 3.5 showed total eradication of the population up to the 50% concentration. The log₁₀ reductions for the 1% test solutions were −1.5 log₁₀ and −5 log₁₀ at 6 and 24 hours, respectively. The Staphylococcus aureus test solutions at pH 5.0 showed concordant results with a −5 log₁₀ reduction at 100% and 50% concentrations and −3.6 log₁₀ and −5 log₁₀ at 10% concentration after 6 and 24 hours, respectively. The 1% concentration test solution achieved a −0.6 log₁₀ reduction.

Growth of Trichophyton rubrum was completely inhibited at the pH 3.5 test solutions at 100% and 50% concentrations. The 10% test solution showed −4 log₁₀ and −5 log₁₀ reductions after 6 and 24 hours, respectively. The 1% concentration solution showed −0.3 log₁₀ and −0.6 log₁₀ reductions after 6 and 24 hours, respectively. The inhibitory effect results of the pH 5.0 test solution correlated with the solution's concentration. The test solution at pH 5.0 showed a −5 log₁₀ reduction after 24 hours at 100% and 50%. The 10% concentration test solution gave a reduction of −0.8 log₁₀ after 24 hours and the 1% test solution achieved a −0.6 log₁₀ reduction. Control testes in sterile water showed no log₁₀ reductions.

### Example 13

### Use of ACPE in the Treatment of Skin Wounds Not Associated with Either Bacterial or Fungal Infections

An ACPE formulation comprising 0.3 g/ml of ACPE in water was tested on 10 human individuals who observed open skin wounds resulting from active diabetes. ACPE was spread on the affected skin twice a day for a period of one week. Within 4 to 5 days after first administration of the ACPE, full healing of the broken skin was observed. The ACPE showed no burning or stinging sensation on the wounded skin and could also be applied on children suffering from similar skin conditions.

### Example 14

### ACPE Activity Against Food-Associated Microorganisms

In this experiment, 300 μL of ACPE solution containing 0.3 g ACPE per 1 ml of water was placed on 13-mm discs, which were positioned in the center of Petri dishes containing various food-associated microorganisms. As may be observed from Table 4, ACPE was shown to inhibit the growth of all 5 microorganisms.

TABLE 4
Inhibition of food-associated bacteria

| Food Bacteria | ACPE Inhibition (diameter in mm) |
|---|---|
| E-Coli 0157 | 16 |
| Staphylococcus aureus | 27 |
| Streptococcus faecalis | 17 |
| Pseudomonas aeruginosa | 17 |
| Salmonella typhimurium | 16 |
| Listeria monocytogenes | 23 |

The ACPE was active against all five strains of Salmonella tested: D-51234 pathogenic, C-51348, C-51119, C-51119 (Chicken run II) and C-51119 (Chicken Spleen). In addition, ACPE was active against the following strains of the Pastorella bacteria: 73204, 77745, 72262, 72784, and 78567.

The ACPE was also active in inhibiting the growth of the following strains of the E-Coli bacteria: 51460, 51461, and 51292.

### Example 15

### ACPE Against Microorganisms in Vegetable and/or Fruit Juice

In these tests, ACPE was added in varying concentrations of 0.1, 0.5, 1, 2.5 and 5%, to various fruit juices such as tomato juice and fresh grapefruit juice. The treated juices were incubated at 20° C. for two weeks, at the end of which period they were tested for the existence of the bacteria. After 12 days, 0.1 μl of each of the juice samples was removed and placed on a Petri dish containing Nutrient agar medium and was tested 12 hours later for the presence of the bacteria. Control studies involved juices containing no preservatives and juices into which Protecta (a brand name of a preservative for juices) was added. Results not shown here indicated that the ACPE inhibited growth of microorganisms similarly to other preservatives tested.

1-90. (canceled)

91. A composition comprising an activated citrus peel extract (ACPE) prepared by an activation method which includes exposure of citrus peels to at least one pathogen, said ACPE comprises at least one of the following: oligosaccharides, short peptides, flavonoid glycosides, fatty acids, and triglycerides.

92. A composition according to claim 91, wherein said ACPE comprises a combination of all of the following: oligosaccharides, short peptides, flavonoid glycosides, fatty acids, and triglycerides.

93. The composition of claim 92, wherein said ACPE comprises at least 30-60% oligosaccharides, 1-10% short peptides, 10-30% flavonoid glycosides, 5-15% fatty acids, and 5-15% triglycerides.

94. The composition according to claim 91, wherein said at least one pathogen is selected from a plant fungal pathogen, a plant bacterial pathogen or a combination thereof.

95. The composition according to claim 94 wherein said plant fungal pathogen is Penicillium digitatum.

96. A dermatological composition comprising: (i) an activated citrus peel extract (ACPE) prepared by an activation method which includes exposure of citrus peels to at least one pathogen, said ACPE comprises 55% oligosaccharides, 5% short peptides, 20% flavonoid glycosides, 10% fatty acids and 10% triglycerides and (ii) a dermatologically acceptable carrier, excipient or diluent.

97. The composition according to claim 96 adapted for the treatment of skin conditions associated with a bacterial or a fungal infection.

98. The composition according to claim 97, wherein said bacterial infection is caused by bacteria selected from Propionibacterium acnes, Entrococcus, hemolytic Streptococci, Staphylococci and M.R.S.A and wherein said fungal infection is caused by a fungus selected from Canis, Trichophyton, Mentagraphtes, Rubrum, Violaceum, Epidermophyton, Icrosporum and Candida.

99. The composition according to claim 97, wherein said skin condition associated with a bacterial infection is selected from acne, cellulites, folliculitis, boils (or carbuncles), staphylococcal scalded skin syndrome, Erysipelas, Erythrasma, Impetigo and Paronychia and wherein said skin condition involving fungal infections is selected from ringworm, Candidiasis, Tinea Pedis and Tinea versicolor.

100. The composition according to claim 96 adapted for the treatment of skin conditions not associated with a bacterial or a fungal infection.

101. The composition according to claim 100, wherein said skin condition is selected from diabetes related skin disorders, skin injuries, dermatitis, bed sores, dry skin, celluses, corns, Keratosis Pilaris, psoriasis, pityriasis rosea and rosacea.

102. The composition of claim 96 formulated as soap, lotion, hand cream, face cream, foot cream or a shampoo.

103. A method for the treatment of a subject suffering from a skin condition, said method comprises contacting the skin of said subject with an effective amount of an activated citrus peel extract (ACPE), said ACPE is prepared by an activation method which includes exposure of citrus peels to at least one pathogen, and comprises at least one or a combination of the following: oligosaccharides, short peptides, flavonoid glycosides, fatty acids, and triglycerides.

104. The method according to claim 103, wherein said ACPE comprises at least 30-60% oligosaccharides, 1-10% short peptides, 10-30% flavonoid glycosides, 5-15% fatty acids, and 5-15% triglycerides.

105. A composition for use in preserving foods, beverages and cosmetics, said composition comprises an activated citrus peel extract (ACPE) prepared by an activation method which includes exposure of citrus peels to at least one pathogen, said ACPE comprising at least one or a combination of the following: oligosaccharides, short peptides, flavonoid glycosides, fatty acids, and triglycerides.

106. The composition according to claim 105 wherein said ACPE comprises 55% oligosaccharides, 5% short peptides, 20% flavonoid glycosides, 10% fatty acids and 10% triglycerides.

107. The composition according to claim 106, wherein said composition is used as an antioxidant.

108. A method for preserving foods, beverages and cosmetics comprising applying to said foods, beverages or cosmetics an effective amount of activated citrus peel extract (ACPE), said ACPE prepared by an activation method which includes exposure of citrus peels to at least one pathogen, and comprising at least one or a combination of the following: oligosaccharides, short peptides, flavonoid glycosides, fatty acids, and triglycerides.

109. The method according to claim 108, wherein said ACPE comprises at least 30-60% oligosaccharides, 1-10% short peptides, 10-30% flavonoid glycosides, 5-15% fatty acids, and 5-15% triglycerides.

110. The method according to claim 109, wherein said ACPE comprises 50-60% oligosaccharides, 3-7% short peptides, 15-25% flavonoid glycosides, 8-12% fatty acids, and 8-12% triglycerides.

111. The method according to claim 110, wherein said ACPE comprises 55% oligosaccharides, 5% short peptides, 20% flavonoid glycosides, 10% fatty acids and 10% triglycerides.

112. A process for the preparation of an activated citrus peel extract (ACPE) comprising:
(i) contacting citrus peels with spores of at least one fungal or bacterial pathogens, said pathogens being a 16 hour to 24 hour old bacteria or a 8 day to 14 old fungus,
(ii) incubating said citrus peels;
(iii) extracting the peels with water, and removing the peels from the aqueous liquid thereby obtaining an aqueous extract;
(iv) filtering said aqueous liquid through a membrane having a cutoff between 800-2000 Da and concentrating the filtrate to obtain said activated citrus peel extract.

113. An activated citrus peel extract (ACPE) obtained by the process of claim 111.
